(19) United States
(12) Patent Application Publication
(10) Pub. No.: US 2012/0083819 A1
(43) Pub. Date: Apr. 5, 2012

(54) External scaffolds for expanding strictures in tubular organs and their use

(76) Inventors: Frank Wang, Flushing, NY (US); Tzu Hsin Feng, Flushing, NY (US)

(21) Appl. No.: 13/200,499

(22) Filed: Sep. 23, 2011

Related U.S. Application Data
(60) Provisional application No. 61/404,418, filed on Oct. 4, 2010.

(51) Int. Cl. A61M 29/00

(52) U.S. Cl. 606/191

(57) ABSTRACT

External scaffolds for expanding a stricture in the lumen of a tubular structure or organ in a mammal are disclosed herein. A method for expanding a stricture in the lumen of a tubular structure or organ in a mammal using the above external scaffolds is also disclosed.

This application claims the benefit of U.S. Provisional Application No. 61/404,418, filed Oct. 4, 2010, the content of which is hereby incorporated by reference in its entirety.

### BACKGROUND OF THE INVENTION

Stricture, or abnormal obstruction, of tubular structures and organs responsible for conducting various agents within the human body often have adverse and devastating effects on normal physiological function. Tubular structures and organs susceptible to stricture formation include, but are not limited to, the esophagus, the pylorus, the small and large intestines, and the bile ducts in the digestive system; the veins and arteries in the circulatory system; the fallopian tubes in the female reproductive system; as well as the ureters in the urinary system.

Strictures of the esophagus can be caused by or associated with gastroesophageal reflux disease (GERD), esophagitis (inflammation of the esophagus), dysfunctional lower esophageal sphincter, disordered motility, lye ingestion, or a hiatal hernia, among others. Strictures can also form after esophageal surgery and other treatments, such as laser therapy or photodynamic therapy. While the area heals a scar forms, causing the tissue to pull and tighten which leads to difficulty in swallowing. Treatment of esophageal strictures usually involves lifestyle and diet changes to reduce GERD. Surgical intervention is a treatment of last resort. An example of surgical treatment of esophageal strictures is bouginage. Bougienage involves passing several tubes of different diameter through the esophagus to stretch the stricture. Alternately, an endoscope containing a deflated balloon at the tip can be inserted into the esophagus and the balloon inflated at the point of the stricture. This process often must be repeated because it does not remove the scar tissue and the esophagus narrows again.

Pyloric stenosis, or abnormal narrowing of the duct (the pylorus) connecting the stomach to the small intestines, is a condition that affects some infants in the first few months of life. Infants suffering from pyloric stenosis exhibit severe vomiting. Pyloric stenosis also occurs in adults where the cause is usually a narrowed pylorus due to scarring from chronic peptic ulceration. This is a different condition from the infantile form, wherein there is narrowing of the pylorus due to enlargement of the muscle surrounding the pylorus, which spasms when the stomach empties. Both pharmacotherapy and surgery can be used to treat pyloric stenosis. When pharmacotherapy is not effective, pyloric stenosis can be treated with surgery wherein the muscle of the pylorus is divided to open up the gastric outlet in a procedure called Ramstedt's procedure.

Intestinal obstruction is a blockage of the small intestine or colon, which makes up part of the large intestines, that prevents food and fluid from passing through. Signs and symptoms of intestinal obstruction include abdominal pain and swelling, nausea, and vomiting. If left untreated, intestinal obstruction can cause the blocked parts of the intestine to die. This tissue death can lead to perforation of the intestine, severe infection and shock. Obstruction in the small intestines can be caused by intestinal adhesions, which are bands of fibrous tissue in the abdominal cavity can form after abdominal or pelvic surgery; hernias, which are portions of intestine that protrude into another part of your body; tumors; inflammatory bowel diseases, such as Crohn's disease; and twisting of the intestine known as volvulus, among other causes. Obstruction of the colon, which makes up large portion of the large intestines, can be caused by colon cancer, diverticulitis, volvulus of the colon, impacted feces, and narrowing of the colon caused by inflammation and scarring, among other causes. Depending on the severity of the obstruction, various treatments may be used. For complete obstruction, in which nothing can pass through the affected area, surgery may be required to relieve the blockage. Surgery typically involves removing the obstruction, as well as any section of the intestine that has died.

The bile ducts are any of a number of long tube-like structures that carry bile. A biliary stricture (a stricture of a bile duct) is often caused by surgical injury to the bile ducts. For example, it may occur after surgery to remove a gallbladder. Other causes of this condition include, but are not limited to, cancer of the bile duct, damage and scarring due to a gallstone in the bile duct, and pancreatitis. The symptoms of a patient suffering from biliary strictures include abdominal pain, chills, fever, pruritis, jaundice, nausea, and vomiting, among others. Treatments may involve surgery or less invasive procedures, such as endoscopic or percutaneous dilation. In some cases, a stent, which is a tiny metal or plastic mesh tube, is placed inside the lumen of the bile duct at the site of the stricture to keep it open.

Blood vessels, which include the arteries and veins, are also susceptible to strictures. In general, arteries carry blood from the heart to other organs in the body while veins carry blood away from the organs to the heart. Causes of arterial strictures include, but are not limited to, atherosclerosis, birth defects, diabetes, iatrogenic causes (e.g. secondary to radiation therapy), infection, inflammation, and ischemia, among others. Treatments of arterial strictures include pharmacotherapy using antiplatelet drug and/or surgery, such as endarterectomy or placement of stents inside the affected artery.

The fallopian tubes of the female reproductive system are also susceptible to obstruction, which can lead to infertility. Difficulty with tubal transport usually occurs because scarring has developed in the fallopian tubes. This scarring can block the agents necessary for a successful fertilization. The scarring resulting in obstruction usually occurs from chronic pelvic inflammatory disease (PID), a ruptured appendix, or abdominal surgery involving infection and subsequent adhesion in the fallopian tubes. Treatments options for opening obstructed fallopian tubes include, but are not limited to, salpingostomy, which involves creating a new opening in the area of the fallopian tube that lies closest to the ovaries; tubal reanastomosis, which involves the removal of the damaged part of the tube and joining of the two healthy ends of the tube through an abdominal incision.

Ureteral strictures, which are obstructions of the ureters in the urinary system, are also devastating to health and quality of life. A ureteral stricture is characterized by a narrowing of the ureteral lumen, which subsequently results in functional obstruction. Ureteral strictures are of particular interest because the widespread use of upper tract endoscopy has led to an increased frequency of iatrogenic ureteral strictures. Thus, even the use of ureteroscopy for management of calculus (kidney stones) can be a cause of ureteral strictures.

The ureter is a muscular tube lined by transitional epithelium that courses from the renal pelvis to the bladder in the retroperitoneum. In both men and women, the ureter courses posterior to the gonadal vessels and anterior to the iliopsoas muscles, crosses the common iliac artery and vein, and enters inferiorly into the pelvis.

Ureteral strictures are typically due to ischemia, or restriction in blood supply, which results in fibrosis in the ureter. A stricture has been defined as ischemic when it follows open surgery or radiation therapy, whereas the stricture is considered non-ischemic if it is caused by spontaneous stone passage or a congenital abnormality. Less commonly, the etiology of ureteral strictures is mechanical, such as the result of poor placement of a permanent suture or surgical clip.

At present, there are no pharmacotherapy options for the management of ureteral strictures. Surgical treatment options for ureteral strictures include balloon dilation, endoureterotomy, ureteral metal stents, such as double “J” pigtail stents, and open surgical management, which includes various treatment options such as psoas hitch, Boari flap, ureteroneocystostomy, transureteroureterostomy (TUU), intestine interposition, renal mobilization, and autotransplant. All open procedures carry an increased risk of morbidity, increased recovery time, and increased hospitalization time compared with endoscopic approaches. For midureteral strictures, a primary ureteroureterostomy may be appropriate for short benign strictures with minimal tension. Proximal ureteral strictures may be managed with ureteropyelostomy if length allows. Also, ureterocalicostomy is useful if the renal pelvis is scarred or intrarenal in location. Long, complex upper tract ureteral strictures have traditionally been managed with nephrectomy, bowel interposition, and autotransplantation. For long, extensive ureteral strictures that are not amenable to repair with urothelium, ileal ureteral substitution may be a satisfactory solution. Laparoscopic and robot-assisted laparoscopic repair may also be used.

As described hereinabove, treatment of strictures of tubular structures and organs in the human body generally require dilation of the affected structure or organ from within the interior of the lumen (eg. balloon dilation, stent placement, etc.) or surgical interventions which carry with them increased risk of morbidity, increased recovery time, and increased hospitalization time. The use of stents is also disadvantageous because stent insertion/removal is intrusive and stent migration can occur wherein the stent migrates away from the site of placement. Stent removal also causes inflammation, which results in decrease in flow. Furthermore, stent encrustation becomes a problem if the stent is not changed frequently. Long-term stent changes may reduce quality-of-life of the patient.

Thus, there is a need for new devices and methods for relieving strictures that has the advantages of being permanent, not involving placement of a device within the lumen of the tubular structure or organ, decreased risk of morbidity, decreased recovery time, and decreased hospitalization time.

Herein, novel external scaffolds for expanding strictures in tubular structures and organs, and methods for their use are disclosed.

### SUMMARY OF THE INVENTION

This invention provides an external scaffold for expanding a stricture in the lumen of a tubular structure or organ in a mammal comprising one or more open frames.

This invention also provides an adjustable external scaffold for expanding a stricture in the lumen of a tubular structure or organ in a mammal, the adjustable external scaffold comprising one or more arcuate assemblies, each arcuate assembly comprising a first semicircular arc and a second semicircular arc pivotally connected at a hinge.

This invention further provides a method for expanding a stricture in the lumen of a tubular structure or organ in a mammal, the method comprising the steps of identifying the location of a stricture in the lumen of a tubular structure or organ in a mammal, placing the scaffold described herein at the site of the stricture, suturing in place the scaffold at the site of the stricture using sutures, and confirming restoration of flow to said tubular structure or organ.

### BRIEF DESCRIPTION OF THE FIGURES

FIG. 1 is a top plan view of an open frame 1 of an external scaffold in accordance with the present invention.

FIG. 2 is a top plan view of an open frame 1w.

FIG. 3 is a top plan view of an open polygonal frame 1v.

FIG. 4 is a top plan view of an open Reuleaux polygonal frame 1x.

FIG. 5 is a top perspective view of an open toroidal frame 1y.

FIG. 6 is a top plan view of an open toroidal frame 1y.

FIG. 7 depicts various shapes that can be revolved around an axis of revolution to produce open toroidal frames.

FIG. 8 is a top perspective view of open toroidal frames generated by revolving various shape around an axis of revolution followed by addition of frame indentations.

FIG. 9 is a top perspective view of an open toroidal frame 1z.

FIG. 10 is a top plan view of an open toroidal frame 1z.

FIG. 11 is a top perspective view of an open toroidal frame 1u.

FIG. 12 is a top plan view of an open toroidal frame 1u.

FIG. 13 is a top plan view of an open toroidal frame 1y showing frame indentation width 15 and frame indentation depth 16.

FIG. 14 is a top perspective view of an open toroidal frame 1y showing frame indentation width 15.

FIG. 15 is a top plan view of an open frame comprising 3 spikes 18.

FIG. 16 is a top perspective view of an open frame comprising 3 spikes, wherein the second segment of each spike is oriented in a direction perpendicular to the scaffold axis.

FIG. 17 is a top perspective view of an open toroidal frame. The scaffold axis 22 is shown.

FIG. 18 is a top perspective view of an external scaffold comprising 2 open frames. The scaffold axis 22 is shown.

FIG. 19 is a top perspective view of another external scaffold comprising 2 open frames. The scaffold axis 22 is shown.

FIG. 20 is a top perspective view of an open frame comprising 3 spikes, wherein the second segment of each spike is oriented in a direction parallel to the scaffold axis.

FIG. 21 is a top perspective view of a protective sleeve suitable for preventing premature penetration of an affected tubular structure or organ by the spikes according to an embodiment of the present invention.

FIG. 22 is a top perspective view of a protective sleeve in use with an external scaffold comprising one open frame, the open frame comprising 3 spikes, wherein the second segment of each spike is oriented in a direction perpendicular to the scaffold axis.

FIG. 23 is a bottom perspective view of a protective sleeve in use with an external scaffold comprising one open frame, the open frame comprising 3 spikes, wherein the second segment of each spike is oriented in a direction parallel to the scaffold axis.

FIG. 24 is a top plan view of an open frame depicting interchangeable and complementary clasping means 23a.

FIG. 25 is a partial side view of interchangeable and complementary clasping means 23a in a disengaged position.

FIG. 26 is a top plan view of an open frame depicting interchangeable and complementary clasping means 23b.

FIG. 27 is a top plan view of an open frame depicting interchangeable and complementary clasping means in a disengaged position.

FIGS. 28 and 29 are side perspective views of an external scaffold comprising 3 open frames.

FIG. 30 is a top plan view along the scaffold axis of an external scaffold comprising 2 open frames, which form a twist angle 30.

FIG. 31 is a side perspective view of another external scaffold comprising 3 open frames.

FIG. 32 is a side perspective view of yet another external scaffold comprising 5 open frames.

FIG. 33 is a top perspective view of yet another external scaffold comprising 2 open frames.

FIG. 34 is a side perspective view of an adjustable external scaffold in accordance with the present invention.

FIG. 35 is a top perspective view of an adjustable external scaffold.

FIG. 36 is an exploded side perspective view of an adjustable external scaffold.

FIG. 37 is a side perspective view of a semicircular arc 32.

FIG. 38 is a side perspective view of a semicircular arc 33.

FIG. 39 shows a tubular structure or organ afflicted by a stricture in a lateral view. Sutures are used to pull and tie the muscular layer to an external scaffold so as to expand the stricture.

FIG. 40 shows a stricture in a tubular structure or organ that has been expanded according to the method of the present invention in a lateral view.

FIG. 41 shows, in a cross-sectional view of the tubular structure or organ, a suitable suturing pattern and sequence for using the external scaffold of the present invention.

FIG. 42 shows, in a cross-sectional view, a stricture in a tubular structure or organ that has been expanded according to the method of the present invention. Additional sutures are also shown.

### DETAILED DESCRIPTION OF THE INVENTION

As used herein, “tubular structures” or “tubular organs” refer to any physiological structure having an essentially hollow and tubular shape capable of carrying fluids and/or agents within a mammal. Tubular structures and organs found in mammals that are susceptible to stricture formation from a variety of different causes or conditions include, but are not limited to, the esophagus, the pylorus, the small and large intestines, and the bile ducts in the digestive system; the veins and arteries in the circulatory system; the fallopian tubes in the female reproductive system; as well as the ureters in the urinary system.

The term “lumen” as used herein, refers to the hollow space or opening of a tubular structure or organ in a mammal. In anatomy, the lumen is lined by a mucosal layer, or mucosa. Surrounding the mucosal layer is generally a muscular layer known as the muscularis. Further details about the anatomy of tubular structures or organs in mammals, particularly humans, can be found in general references available to the person of ordinary skill in the medical arts, for example, Saladin, K. S. Anatomy & Physiology: The Unity of Form and Function, 5th edition, 2010, McGraw-Hill, the entirety of which is hereby incorporated by reference.

The term “mammals” as used herein, refer to air-breathing vertebrate animals characterized by the possession of hair, three middle ear bones, and mammary glands functional in mothers with young. Examples of mammals include, but are not limited to, humans; baboons and other primates; pet animals such as dogs and cats; laboratory animals such as rats and mice; and farm animals such as horses, sheep, and cows. The present scaffolds described herein may be used to relieve strictures in any of the foregoing tubular structures or organs in any mammal suffering from a stricture or multiple strictures. In an embodiment, the mammal is human.

This invention provides an external scaffold for expanding a stricture in the lumen of a tubular structure or organ in a mammal comprising one or more open frames. Each open frame comprises a plurality of frame indentations, an inward-facing surface, an outward-facing surface, and an opening between opposite first and second ends.

As used herein, the term “open frame” refers to a substantially planar and rigid shape that is not closed.

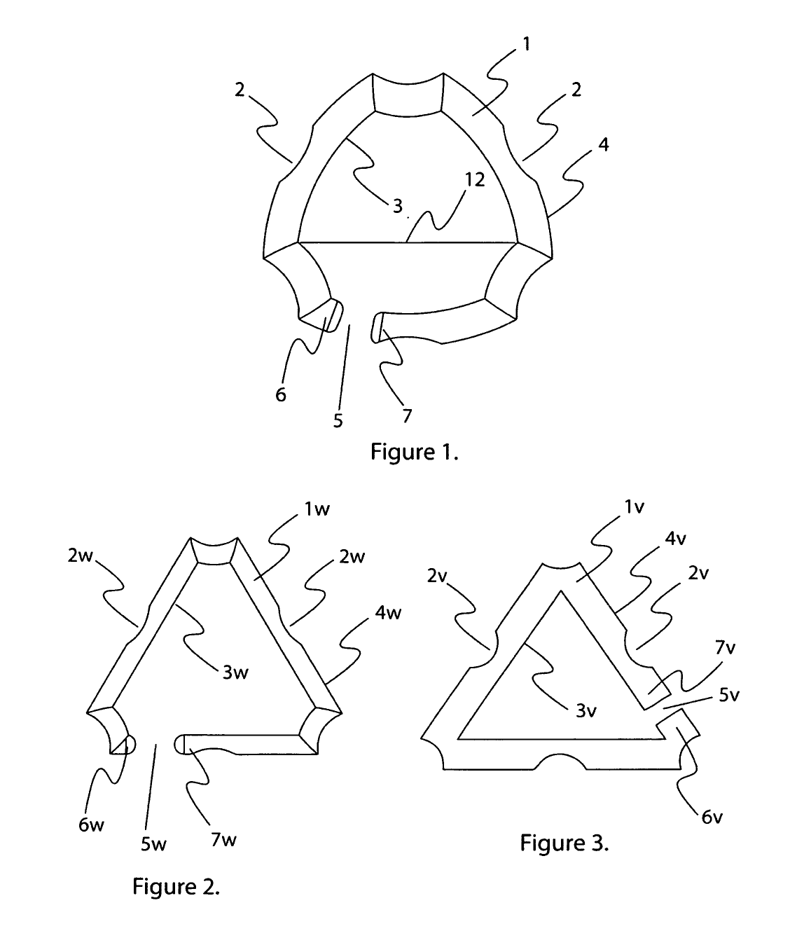

An example of an open frame is illustrated in FIG. 1. Open frame 1 comprises a plurality of frame indentations 2, an inward-facing surface 3, an outward-facing surface 4, and an opening 5 between opposite first and second ends 6 and 7, respectively.

In another example, illustrated in FIG. 2, open frame lw comprises a plurality of frame indentations 2w, an inward-facing surface 3w, an outward-facing surface 4w, and an opening 5w between opposite first and second ends 6w and 7w, respectively.

In an embodiment, the one or more open frames are polygonal or toroidal frames, or a combination thereof.

The term “polygon” or “polygonal”, as used herein, refers to a substantially planar shape composed of a finite sequence of segments, called edges or sides, and the points where two edges meet are the polygon's vertices or corners.

Herein, an equiangular polygon is a polygon wherein all its sides are straight and the corner angles are equal. A polygon is cyclic when all corners lie on a single circle. A polygon is equilateral when all edges are straight and have the same length. A regular polygon is one that is both cyclic and equilateral.

The term “Reuleaux polygon”, as used herein, refers to a polygonal shape having an odd-number of curved sides and has constant width. In other words, the distance from a vertex to an opposite side is constant.

A polygonal frame may be any polygon shape. Preferably, the polygonal frame is a polygon that is symmetric. When the polygonal frame is symmetric, the stricture of an affected tubular structure or organ can easily be expanded in a symmetrical manner.

In an embodiment, the external scaffold comprises one or more open polygonal frames, and each open polygonal frame is independently selected from the group consisting of a regular polygon and a Reuleaux polygon.

An example of an open regular polygon frame is open polygonal frame 1v, illustrated in FIG. 3. Open polygonal frame 1v comprises a plurality of frame indentations 2v, an inward-facing surface 3v, an outward-facing surface 4v, and an opening 5v between opposite first and second ends 6v and 7v, respectively.

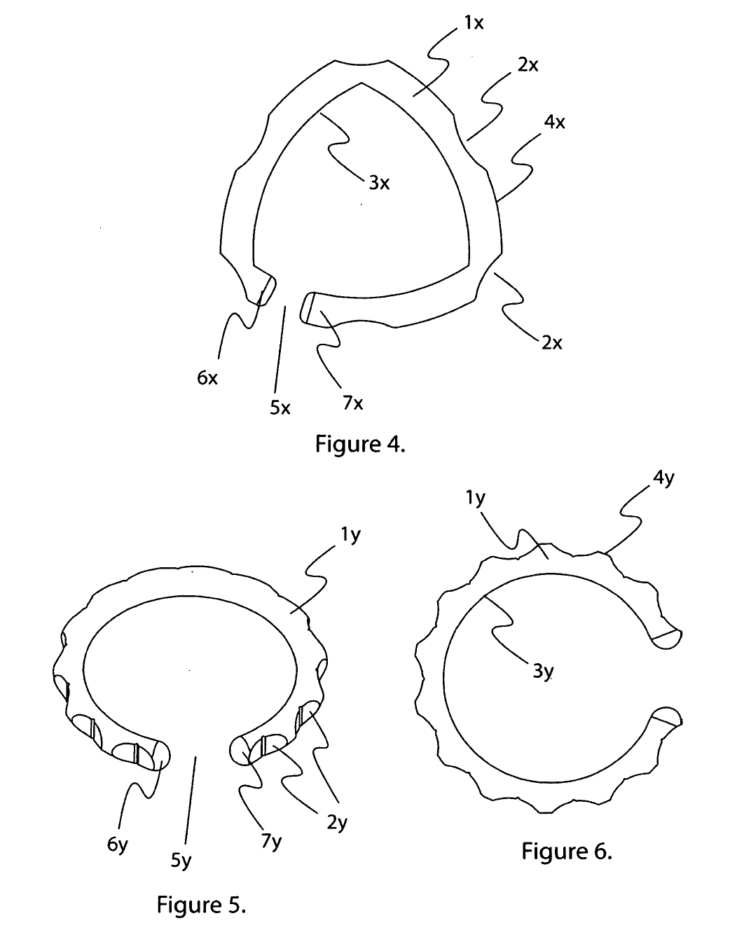

An example of an open Reuleaux polygon frame is open polygonal frame 1x, illustrated in FIG. 4. Open polygonal frame 1x comprises a plurality of frame indentations 2x, an inward-facing surface 3x, an outward-facing surface 4x, and an opening 5x between opposite first and second ends 6x and 7x, respectively.

The term “toroid” or “toroidal”, as used herein, refers to an annular shape that is generated by revolving a plane geometrical figure, including, but not limited, a circle, a polygon, or the like, about an axis external to that figure which is parallel to the plane of the figure and does not intersect the figure. For example, a circle revolved about an axis external to that circle which is parallel to the plane of the circle and does not intersect the circle, generates a torus (doughnut shape).

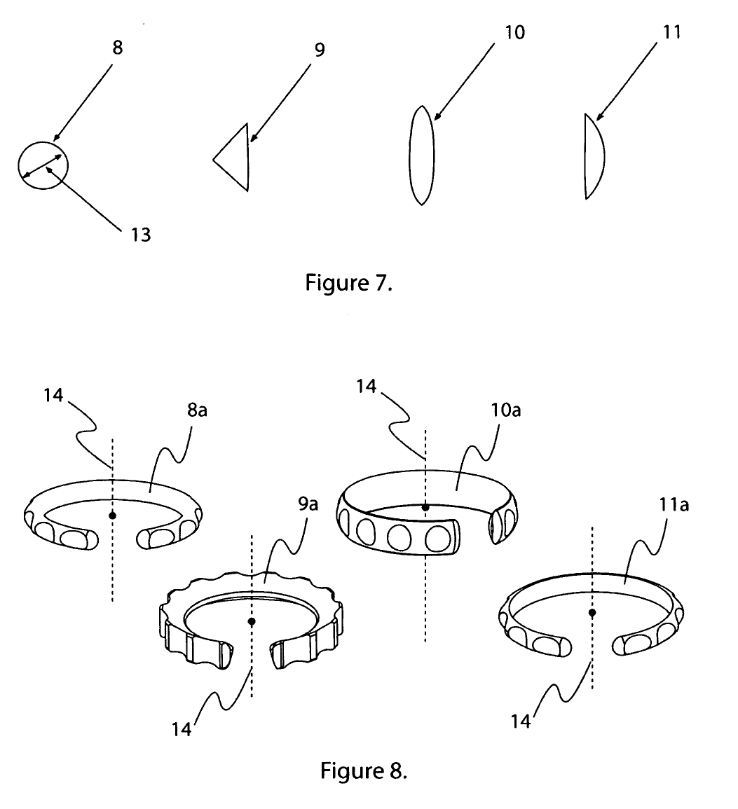

An example of an open toroidal frame is open frame 1y, illustrated in FIGS. 5 and 6. Open frame 1y comprises a plurality of frame indentations 2y, an inward-facing surface 3y, an outward-facing surface 4y, and an opening 5y between opposite first and second ends 6y and 7y, respectively.

Each toroidal frame of the external scaffold may be a toroid generated by revolving any plane geometrical figure about an axis external to that figure which is parallel to the plane of the figure and does not intersect the figure. Herein, such an axis is referred to as the axis of revolution. A toroidal frame generated by revolving any planar geometrical figure is suitable. Preferably, the toroidal frame is a toroid generated by revolving a circle 8, a triangle 9, an oval 10, or an half-oval 11, as illustrated in FIG. 7.

Thus, revolution of circle 8 around an axis of revolution 14 followed by addition of frame indentations, produces open toroidal frame 8a, illustrated in FIG. 8. Revolution of triangle 9 around an axis of revolution 14 followed by addition of frame indentations, produces open toroidal frame 9a. Revolution of oval 10 around an axis of revolution 14 followed by addition of frame indentations, produces open toroidal frame 10a. Revolution of half-oval 11 around an axis of revolution 14 followed by addition of frame indentations, produces open toroidal frame 11a.

The ordinarily skilled artisan will realize that the cross-sectional shape of a toroidal frame is the same as the shape revolved to form it. Such toroidal frames provide enhanced relief for the sutures tied onto them, decreasing risk of wear and breakage of the sutures.

Each open frame has an inner frame width. The inner frame width, as used herein, is defined as the shortest distance between the two farthest points on the inward-facing surface of a frame without traversing any part of the frame. For example, in FIG. 1, the inner frame width 12 is the distance between the two farthest points on the inward-facing surface of a frame without traversing any part of the frame. In an open toroidal frame, the inner frame width is the inner diameter. The inner frame width can be any width so long as it is large enough to expand the stricture of an affected tubular structure or organ. It is important for the frame width to be larger than the diameter of the affected tubular structure or organ itself so as to facilitate proper expansion of the stricture. Preferably, the inner frame width is between about 0.25 inches and about 2.5 inches. More preferably, the inner frame width is between about 0.25 inches and about 0.75 inches.

Each open frame has a cross-sectional width. For example, in an open toroidal frame, the cross-sectional width 13 is illustrated in FIG. 7. The cross-sectional width, as used herein, is defined as the shortest distance between a point on the inward-facing surface and the outward-facing surface while traversing the frame along a cross-sectional plane that does not intersect any part of a frame indentation. Preferably, the cross-sectional width is between about 0.065 inches and about 0.25 inches. If the cross-sectional width is less than 0.065 inches, the frame may be too weak to support sutures tied to it. If the cross-sectional width is larger than 0.065 inches, a longer length of sutures may be needed to tie the affected tubular structure or organ to the frame.

Each open frame of the external scaffold comprises a plurality of frame indentations. Examples of frame indentations are frame indentations 2, illustrated in FIG. 1. The frame indentations prevent sliding of the sutures on the frame. Furthermore, the frame indentations ensure that the sutures are in the proper and symmetric position during and after all sutures are in place. The frame indentations also prevent the sutures from sliding onto one another, thereby collapsing the affected tubular structure or organ. The frame indentations can be any indentation appearing on the surface of the frame. Preferably, the frame indentations are arc-shaped, V-shaped, or a combination thereof. More preferably, the frame indentations are each a combination of an arc-shaped and a V-shaped groove so as to securely hold a suture in place and to prevent post-operative migration of the suture.

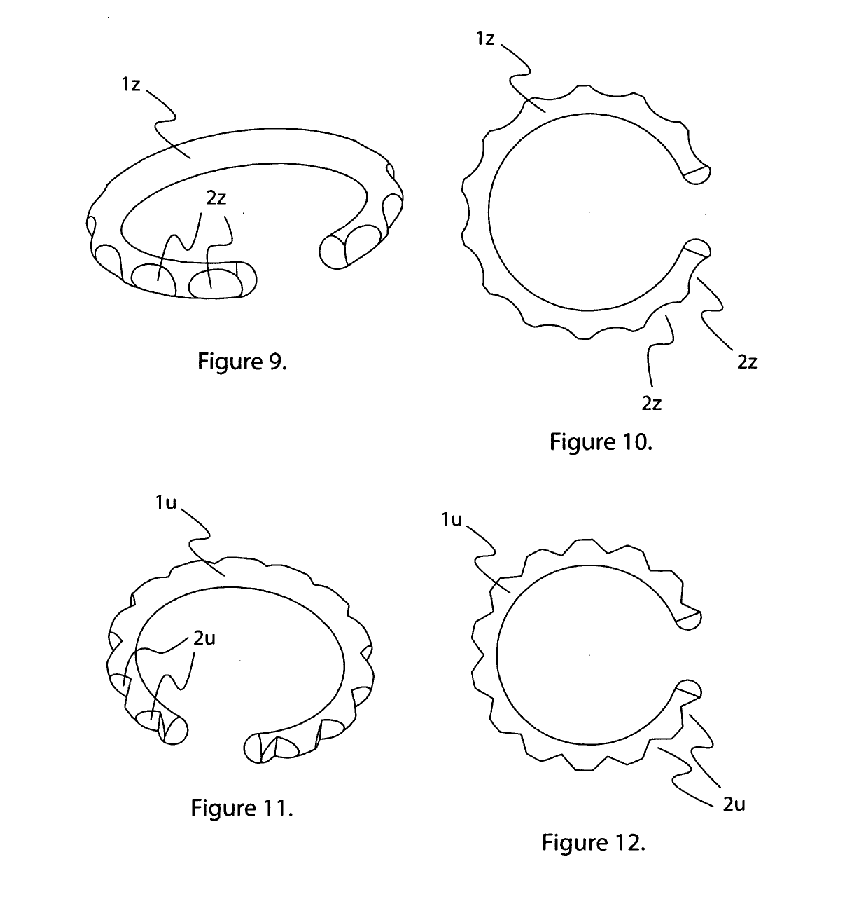

Examples of arc-shaped frame indentations are frame indentations 2z on open frame 1z, illustrated in FIGS. 9 and 10. Examples of V-shaped frame indentations are frame indentations 2u on open frame 1u, illustrated in FIGS. 11 and 12. Examples of frame indentations combining an arc shape and a V shape are frame indentations 2y on open frame 1y, illustrated in FIGS. 5 and 6.

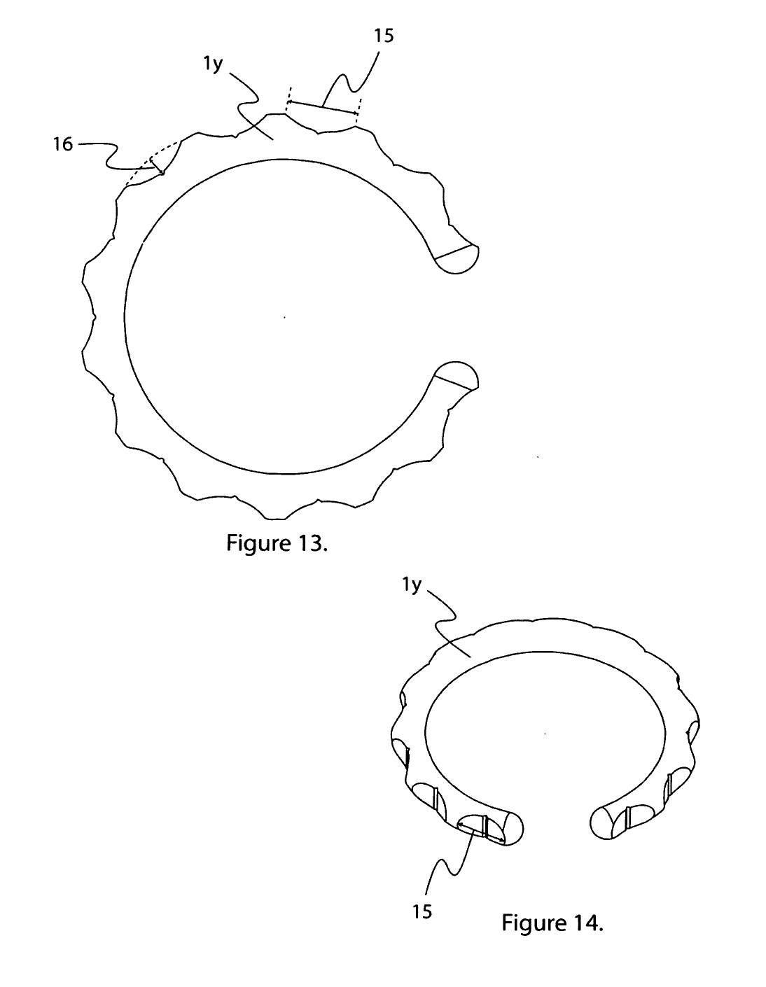

Each frame indentation is characterized by a frame indentation width and frame indentation depth. The frame indentation width refers to the width of the frame indentation from left to right when the frame is held in a horizontal orientation. The frame indentation depth refers to the depth of the frame indentation. For example, open frame 1y has frame indentations, each characterized by a frame indentation width 15 and frame indentation depth 16, as shown in FIGS. 13 and 14. Preferably, each frame indentation has a frame indentation width between about 0.065 inches and about 0.125 inches. Preferably, the frame indentation depth does not exceed one-half of the cross-sectional width of the open frame.

Each open frame of the external scaffold of the present invention has an opening between opposite first and second ends. For example, referring to FIG. 1, open frame 1 has an opening 5 between opposite first and second ends 6 and 7, respectively. The opening allows for placement of the frame around a tubular structure or organ at the site of stricture to be expanded. The opening width, which is defined as the distance between the opposite first and seconds, can be any width as long as the affected tubular structure or organ can fit through the opening. Preferably, the opening between first and second ends has an opening width between about 0 inches and about 0.25 inches. More preferably, the opening width is between about 0.0625 inches and about 0.25 inches. Most preferably, the opening width is about 0.125 inches. If the frame is deformable, i.e. made of flexible material and capable of being flexed, the opening may be mechanically widened, thereby increasing the opening width, to allow the affected tubular structure or organ to pass through. Subsequently, the opening can be mechanically narrowed, thereby reducing the opening width, to reduce the probability of the tubular structure to pass through post-operatively.

In an embodiment of the external scaffold, the external scaffold comprises one open toroidal frame, wherein said toroidal frame is a torus frame, said inner frame width is 0.5 inches and said opening width is 0.125 inches.

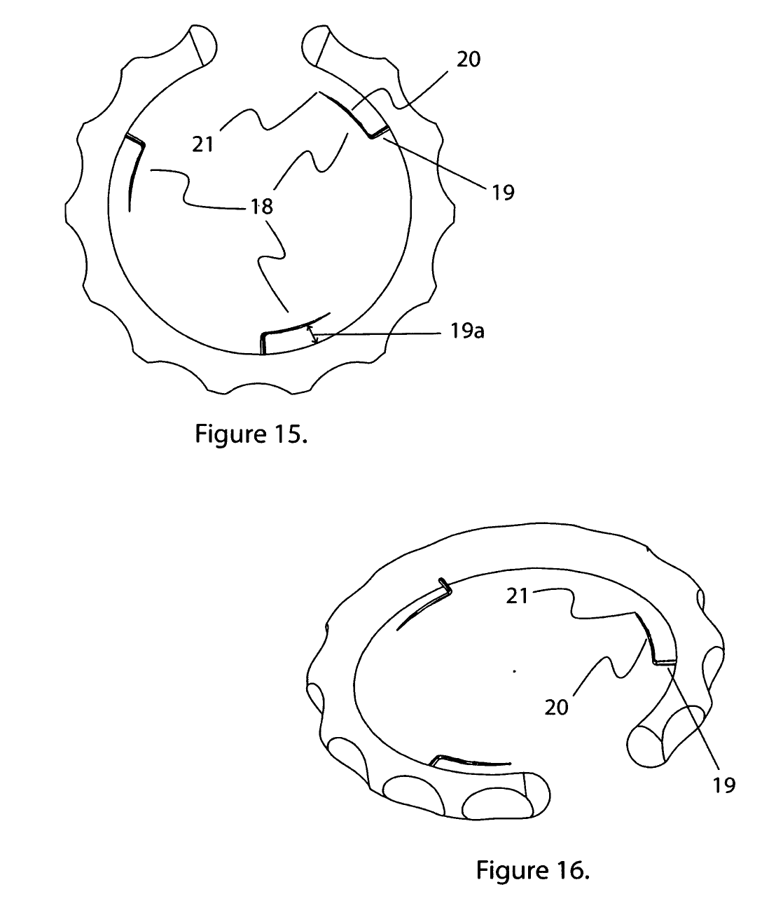

In an embodiment of the invention, the inward-facing surface of each frame comprises 3 or more spikes protruding from said inward-facing surface. For example, referring to FIGS. 15 and 16, the inward-facing surface of each frame comprises 3 or more spikes 18 protruding from said inward-facing surface. Each spike comprises a first segment 19 connected directly to the inward-facing surface and is perpendicular to the scaffold axis. The length 19a of the first segment 19 should not exceed the thickness of the muscle layer of the affected tubular structure or organ. The thickness of the affected tubular structure or organ depends on the identity of the affected tubular structure or organ and can be identified by one having ordinary skill in the medical arts. A second segment 20 is connected directly to the end of the first segment that is not connected to the inward-facing surface and the angle formed between said first and second segment is about 90°. Furthermore, the second segment comprises a needlepoint 21 at the end not connected to the first segment. Preferably, the inward-facing surface of each open frame comprises 3 spikes.

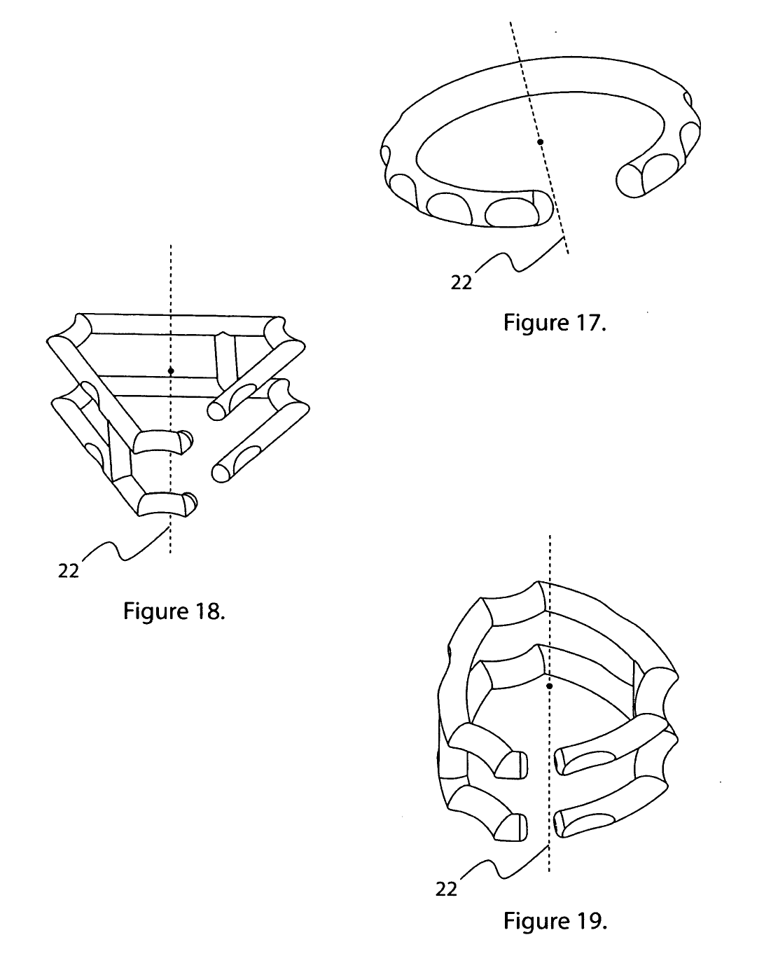

The term “scaffold axis”, as used herein, refers to an axis that is perpendicular to the planes containing each open frame. FIGS. 17-19 illustrates the position of the scaffold axis in several open frame examples. As illustrated in FIG. 17, in a toroidal frame, the scaffold axis 22 is the same as the axis of revolution. In a regular or Reuleaux polygonal frame, the scaffold axis 22 runs through a point equidistant from every vertex that lies on the plane containing a given open frame.

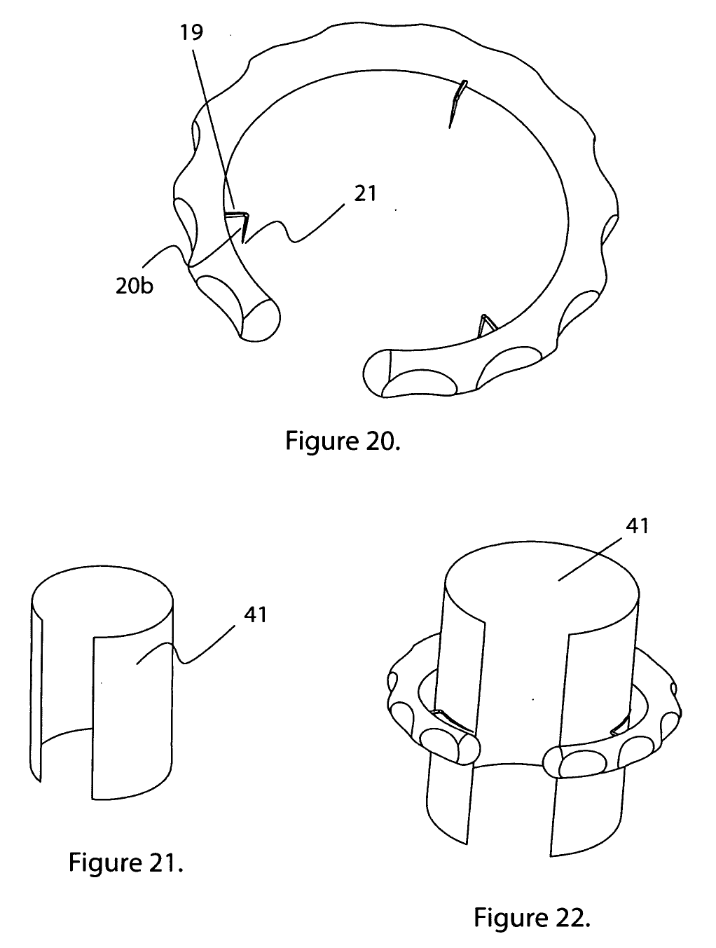

The second segment of each spike can be oriented in a direction parallel or perpendicular to the scaffold axis. In an embodiment, second segment is oriented in a direction parallel to the scaffold axis. For example, as seen in FIG. 20, second segment 20b is oriented in a direction parallel to the scaffold axis. In another embodiment, the second segment is oriented in a direction perpendicular to the scaffold axis. For example, referring to FIG. 15, second segment 20 is oriented in a direction perpendicular to the scaffold axis. In this embodiment, the second segment is curved such that it follows the curve of the inward-facing surface of the open frame.

The presence of the 3 or more spikes on each open frame allows a medical practitioner to secure the external scaffold to a temporarily expanded tubular structure or organ, for example by balloon dilation, prior to suturing, thereby increasing the ease of suturing the external scaffold to the tubular structure. When the second segment of each spike is oriented in a direction parallel to the scaffold axis, the medical practitioner presses the external scaffold along the length of the tubular organ, thereby allowing the spikes to penetrate and engage the muscular layer of the tubular organ. When the second segment of each spike is oriented in a direction perpendicular to the scaffold axis, the medical practitioner rotates the external scaffold around the tubular organ, thereby allowing the spikes to penetrate and engage the muscular layer of the tubular organ.

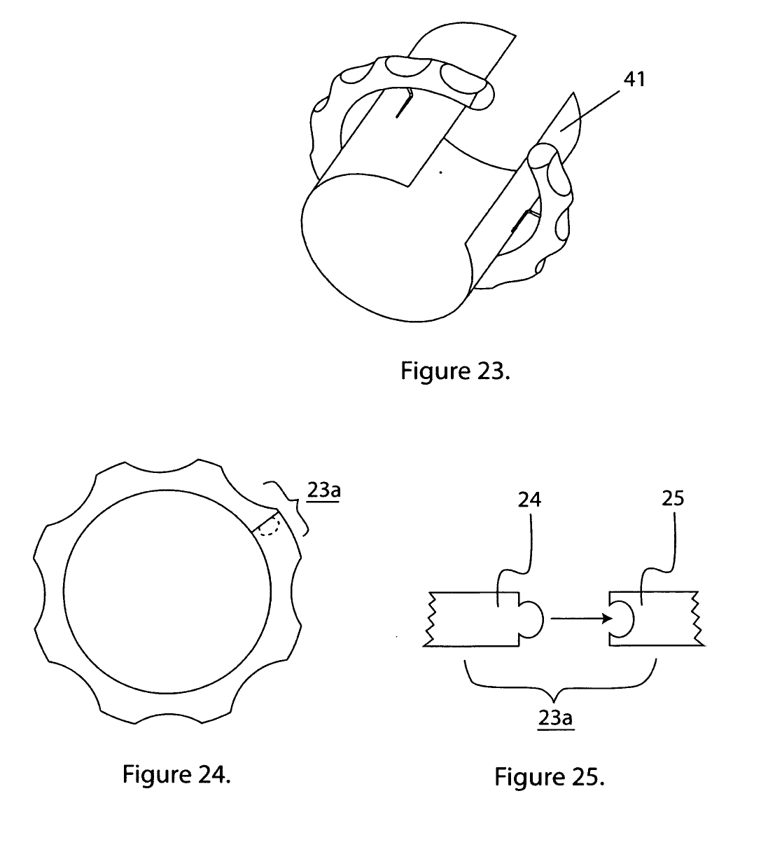

When the external scaffold contains 3 or more spikes, a removable plastic sleeve may be used to prevent premature grasping on the affected tubular structure or organ by the spikes. For example, in FIGS. 21-23, when the external scaffold contains 3 or more spikes, a removable plastic sleeve 41 may be used to prevent premature penetration of the affected tubular structure or organ by the spikes.

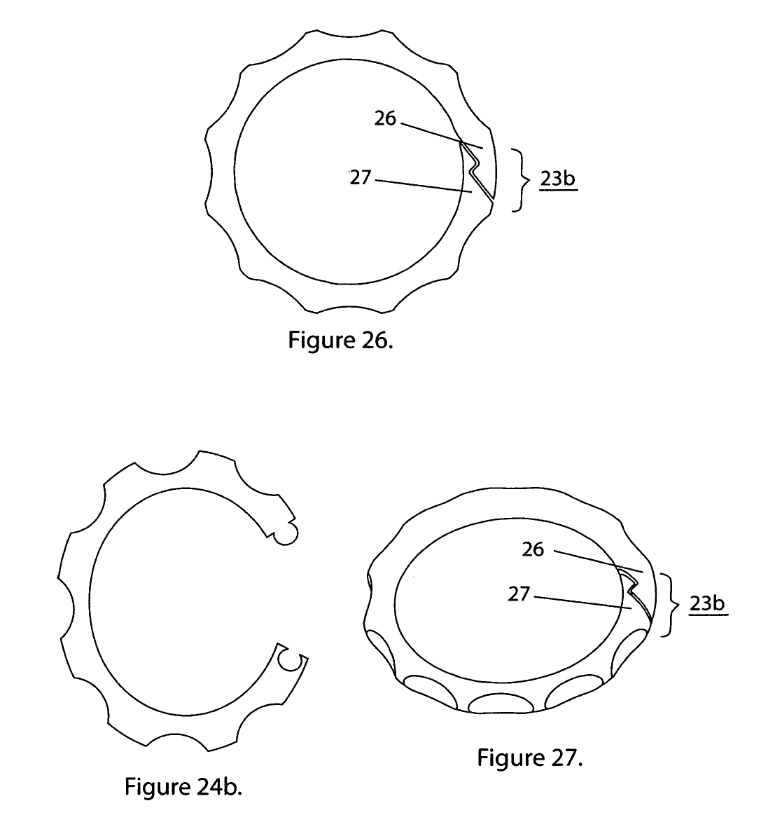

In an embodiment, the opposite first and second ends of the one or more open frames further comprise interchangeable and complementary clasping means capable of being engaged in a locked position. An example of interchangeable and complementary clasping means is interchangeable and complementary clasping means 23a, shown in FIG. 24, wherein the first end is equipped with a “male” protrusion 24 to be mated with the opposite second end of each frame with a complementary “female” cavity 25, shown in FIG. 25, where they are “snapped” together. Another example of interchangeable and complementary clasping means is interchangeable and complementary clasping means 23b, shown in FIG. 26, wherein the first end is equipped with a step shape 26 complementary with the step shape 27 of the opposite second end such that the opposite first and second ends click and lock when brought together. If the frame is deformable, the opening may be mechanically widened, thereby unclasping the clasping means, to allow the affected tubular structure or organ to pass through, as seen in FIG. 24b. Subsequently, the opening can be mechanically narrowed, thereby engaging the clasping means, to close the external scaffold around the tubular structure, as seen in FIG. 24.

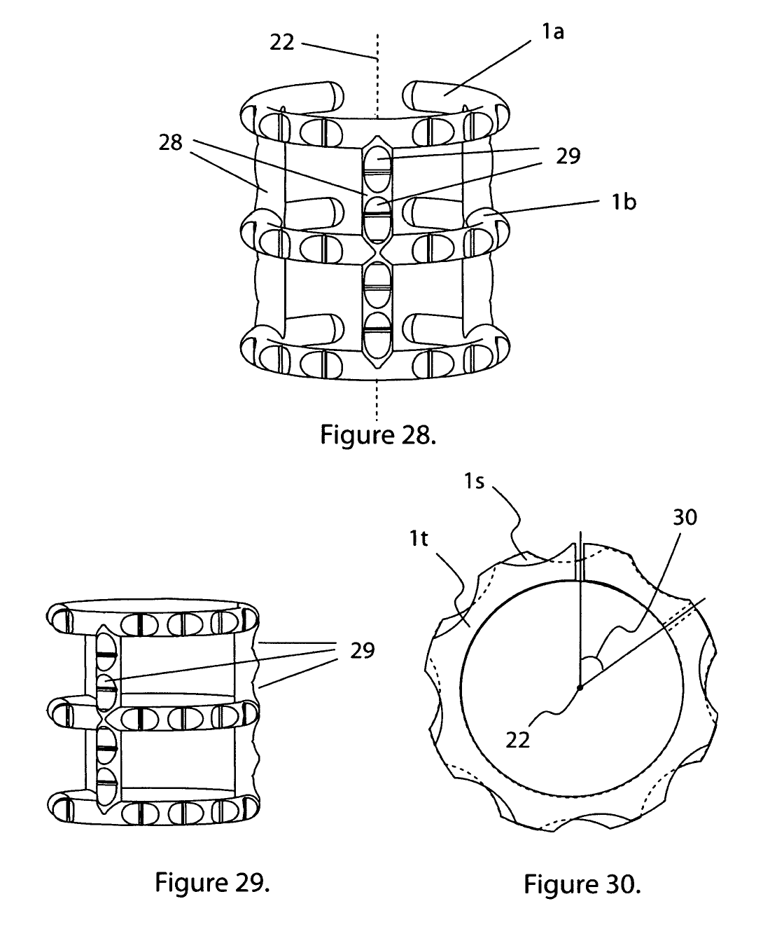

In an embodiment of the invention, the external scaffold comprises two or more open frames. For example, an open frame 1a is juxtaposed to an adjacent open frame 1b along the scaffold axis 22, as shown in FIG. 28. In such a configuration, each open frame is separated from an adjacent open frame by one or more cylindrical spacers. In an external scaffold comprising two or more open frames, there can be any number of cylindrical spacers between adjacent frames. Preferably, the number of cylindrical spacers between adjacent frames is in the range of 1 to 10. When there are more than 10 cylindrical spacers, the external scaffold becomes cumbersome to install as the space between spacers become smaller, preventing efficient suturing. For example, in FIG. 28, cylindrical spacers 28 separate adjacent open frames 1a and 1b. The cylindrical spacers comprise a plurality of spacer indentations to provide supports for additional sutures. The spacer indentations can be any indentation appearing on the surface of the spacer. Preferably, the spacer indentations are arc-shaped, V-shaped, or a combination thereof. More preferably, the spacer indentations are each a combination of an arc-shaped and a V-shaped groove so as to securely hold a suture in place and to prevent post-operative migration of the suture. For example, in FIGS. 28 and 29, spacer indentations 29 are each a combination of an arc shape and a V shape.

The use of two or more open frames in the external scaffold of the present invention allows for customization of the scaffold. It is sectional and has various geometries as described hereinabove so as to allow for adaptation to the length and severity of strictures found in an afflicted mammal.

In an embodiment, the two or more open frames are open polygonal or toroidal frames, or a combination thereof.

In an embodiment, the external scaffold comprises 2 or more open toroidal frames, wherein each open toroidal frame is an open torus frame.

In another embodiment, the opposite first and second ends of each open torus frame further comprise interchangeable and complementary clasping means capable of being engaged in a locked position.

When the external scaffold comprises two or more open frames, each open frame forms a twist angle with an adjacent open frame.

Herein, the term “twist angle” refers to the angle formed between the opening of an open frame with the opening of an adjacent open frame when viewed along the scaffold axis. For example, in FIG. 30 (frame indentations removed for clarity), the twist angle 30 is the angle formed between the opening of an open frame 1s with the opening of an adjacent open frame 1t when viewed along the scaffold axis Preferably, the twist angle of adjacent frames is between about 0° and about 90°. If the twist angle is too large, placement of the external scaffold around a tubular structure or organ becomes difficult.

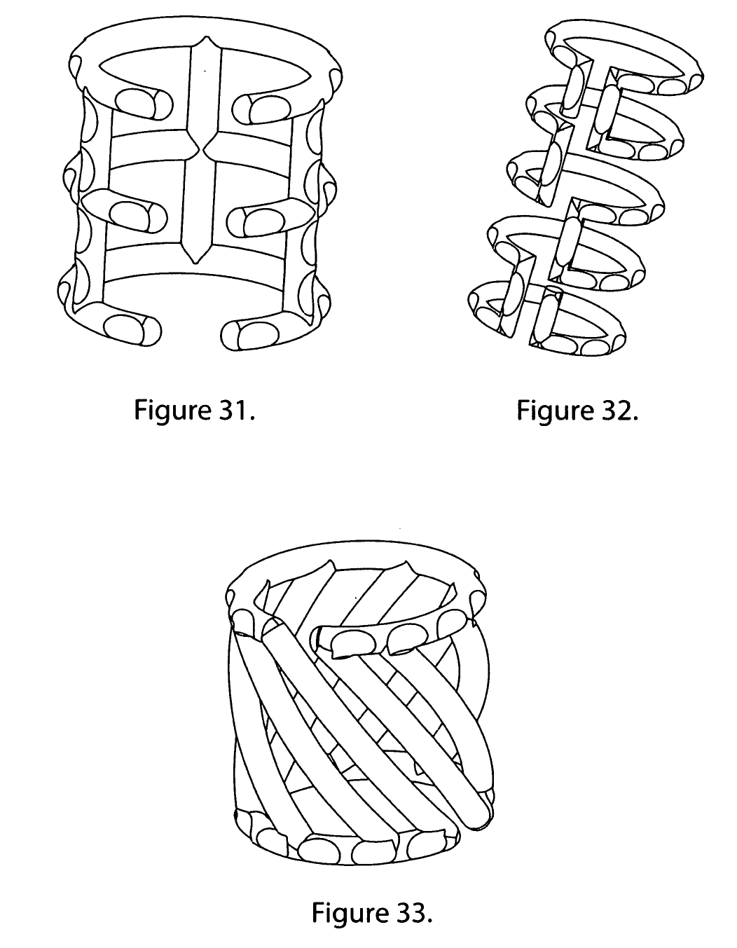

In yet another embodiment, the external scaffold comprises 3 open torus frames, wherein each open torus frame is separated from an adjacent open torus frame by 3 cylindrical spacers, and wherein each open torus frame forms a twist angle of 0° with an adjacent open torus frame, as shown in FIG. 31.

In an embodiment, the external scaffold comprises 5 open torus frames, wherein each open torus frame is separated from an adjacent open torus frame by 1 cylindrical spacer, and wherein each open torus frame forms a twist angle of 0° with an adjacent open torus frame, as shown in FIG. 32.

In yet another embodiment, the external scaffold comprises 2 open torus frames, wherein each open torus frame is separated from an adjacent open torus frame by 9 cylindrical spacers, and wherein each open torus frame forms a twist angle of about 90° with an adjacent open torus frame, as shown in FIG. 33.

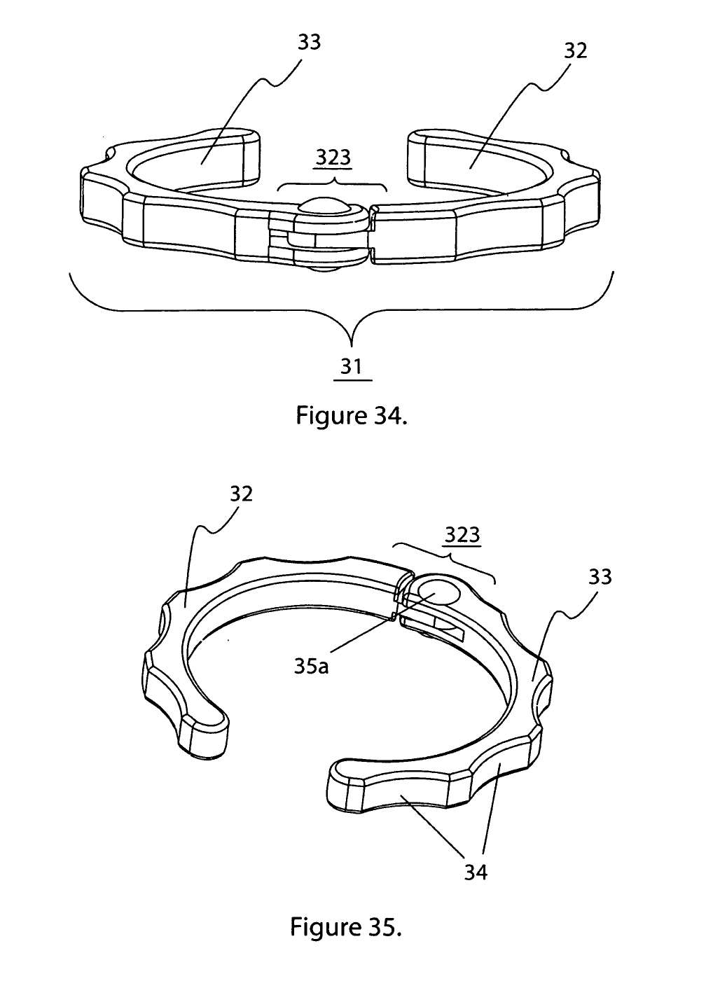

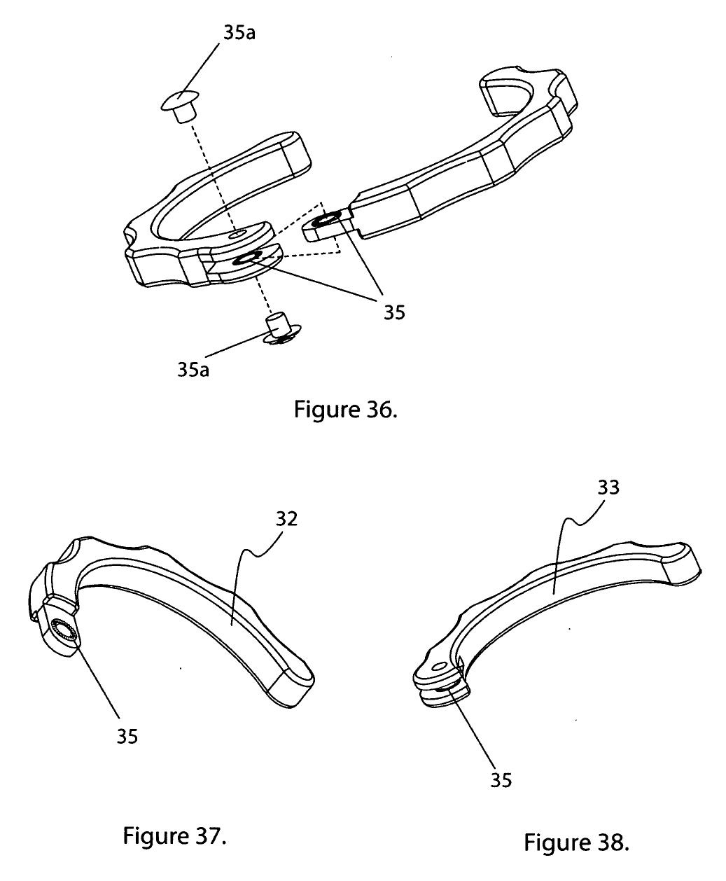

This invention also provides an adjustable external scaffold for expanding a stricture in the lumen of a tubular structure or organ in a mammal, the adjustable external scaffold comprising one or more arcuate assemblies. When the adjustable external scaffold comprises more than one arcuate assembly, cylindrical spacers separate adjacent arcuate assemblies. An example of an arcuate assembly is shown in FIGS. 34-38. For example, an arcuate assembly 31 comprises a first semicircular arc 32 and a second semicircular arc 33 pivotally connected at a hinge 323. The first semicircular arc 32 and the second semicircular arc 33 of each arcuate assembly each comprise a plurality of arc indentations 34 on the outward-facing surface. The hinge comprises a rivet 35a and clicking means 35 whereby the first semicircular arc and the second semicircular are held in place in the absence of force and capable of opening or closing in the presence of force. As illustrated in FIGS. 37 and 38, clicking means 35 comprises complementary teeth by which the first semicircular arc 32 and second semicircular arc 33 are engaged. In the absence of force, the clicking means 35 produces enough friction to hold the arcuate assembly in a fixed shape. In the presence of force, arcuate assembly 31 can be opened or closed.

The external scaffolds of the present invention may be made by manufacturing methods known to those having ordinary skill in the manufacturing arts. Examples of methods suitable for producing the external scaffolds of the present invention include, but are not limited to, casting methods, molding methods, forging methods, and machining methods, such as CNC machining.

The external scaffolds of the present invention can be made of any material suitable for long-term use in a mammal. Examples of suitable materials of manufacture include, but are not limited to, surgical alloy, stainless steel, titanium-alloy, steel-alloy, or surgical plastic.

Hereinafter, methods of using the scaffolds of the present invention are described.

This invention provides a method for expanding a stricture in the lumen of a tubular structure or organ in a mammal, the method comprising the steps of identifying the location of a stricture in the lumen of a tubular structure or organ in a mammal, placing the scaffold described hereinabove at the site of said stricture, suturing in place said scaffold at the site of said stricture using sutures, and confirming restoration of flow to said tubular structure or organ.

Identification of the location of a stricture in the lumen of a tubular structure or organ in a mammal is preferably achieved by injecting a contrast dye near the lumen of a tubular structure or organ in a mammal, observing the flow of the contrast dye through said tubular structure or organ using a fluoroscopy x-ray apparatus, and noting the location of a stricture in said tubular structure or organ. Herein, the term “contrast dye” can be used interchangeably with “contrast agent”, and refers to any type of medical contrast medium used to improve the visibility of internal bodily structures in X-ray medical imaging techniques, including, but not limited to fluoroscopy. Examples of suitable contrast dyes include, but are not limited to, diatrizoate, metrizoate, ioxaglate, iopamidol, iohexol, ioxilan, iopromide iodixanol, barium salts, including barium sulfate, and the like. One having ordinary skill in the art will be able to choose the proper contrast dye for the tubular structure or organ to be examined.

For example, when the tubular structure or organ to be examined is a ureter, the patient is first placed in a prone position. An antegrade percutaneous nephrostomy tube is introduced into the kidney. The patient is then placed on his back. A contrast dye is introduced via the nephrostomy tube into the kidney and fluoroscopy X-ray is used to visualize the ureter to determine the exact location of the stricture. Alternatively, the contrast dye is introduced intravenously without a nephrostomy tube. A retrograde pyelogram may alternatively be performed wherein a catheter is inserted into the urethra, through the bladder, and into the ureter, where the contrast agent is the injected.

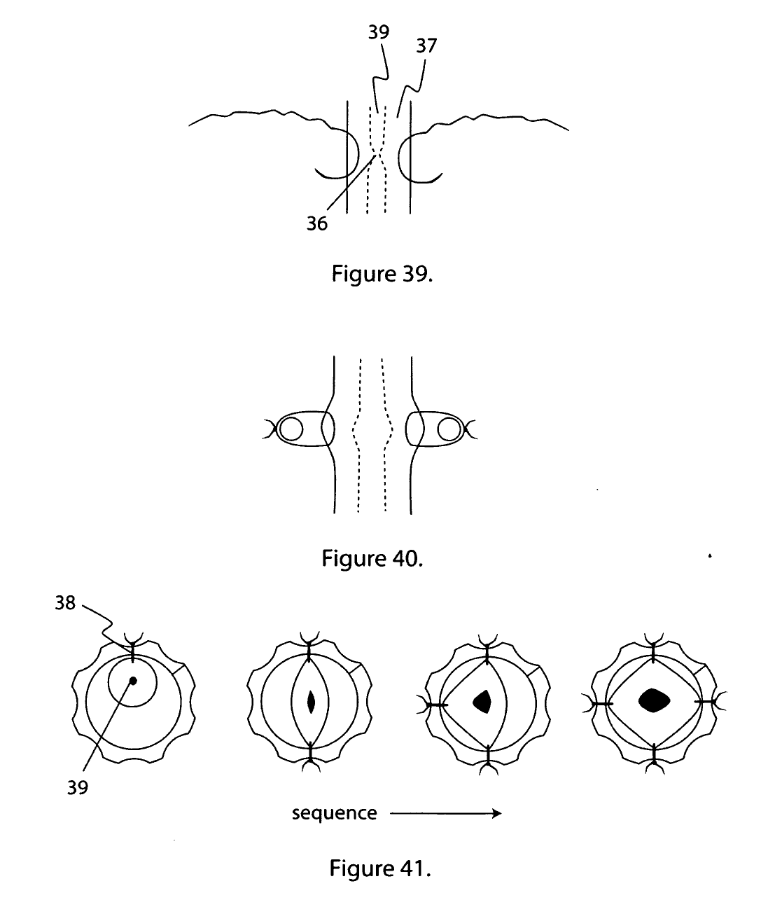

Subsequently, the scaffold described hereinabove is placed at the site of the stricture. For example, placing the scaffold is achieved by providing the scaffold and placing the scaffold over the site of stricture 36 in the tubular structure or organ, and connecting the muscle layer 37 of the tubular structure or organ to the scaffold, as shown in FIGS. 39 and 40. Placement may be done laparoscopically, either manually or through robot assistance. The scaffold is then initially secured at the stricture site by making one initial suture, as shown in FIG. 41. It is crucial that the initial suture, and all subsequent sutures, do not perforate into the lumen 39 of the tubular structure or organ, as perforation into the lumen may lead to mucosal damage, leakage, and subsequent fibrosis. Any type of suture is suitable for use in the present method as long as it does not absorb and become part of the tissue being sutured. Preferably, non-absorbent sutures are used, such as that typically used in micro-surgeries. Examples of non-absorbent sutures include, but are not limited to, silk, cotton, nylon, wire, dacron, linen, silkworm gut, mesh, and tantalum.

For example, when the tubular structure or organ to be examined is a ureter, trocar incisions are made through the abdomen to facilitate laparoscopic instruments, which can be manual, robotic, or both. Once the suspected stricture is located, the scaffold described hereinabove is introduced into the abdomen through the operating ports. Subsequently, the scaffold is placed over the ureter through its opening. An initial suture is placed on the muscular layer of the ureter and tied onto the scaffold to hold it in place.

When the external scaffold contains 3 or more spikes as described hereinabove, a removable plastic sleeve may be used to prevent premature penetration of the affected tubular structure or organ by the spikes during the step of placing the scaffold around the stricture site. Once the external scaffold is in the desired position, the plastic sleeve is removed to expose the spikes, which are then made to penetrate and engage the muscular layer of the affected tubular structure or organ, either by rotating or pushing down along the length of the affect tubular structure or organ. In this situation, it is necessary to use a balloon dilator catheter to expand the stricture and immobilize the external scaffold at the stricture site for suturing. Subsequently, the initial suture is made and the tubular structure or organ is tied onto the scaffold to secure it in place.

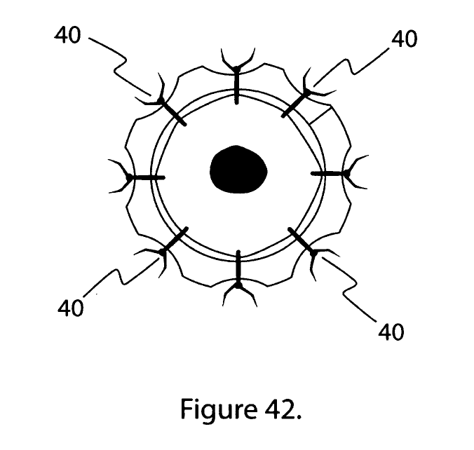

Following the initial suture, subsequent suturing is done to secure the scaffold in place at the site of the stricture. Subsequent sutures are made in a substantially symmetrical pattern around the scaffold. Any symmetrical pattern wherein the sutures are substantially equidistant from one another around each open frame of the external scaffold is suitable. Such a pattern ensures symmetrical suturing around the exterior of the strictured tubular structure, which creates equal pulling force around the tubular structure or organ. For example, sutures are applied at the positions of 12-, 6-, 9-, and 3-o'clock, as shown in FIG. 41. Additional sutures 40 can be introduced symmetrically around the scaffold, as shown in FIG. 42, if needed. As described, it is crucial that the sutures do not perforate the lumen of the tubular structure or organ, as perforation of the lumen may lead to mucosal damage, leakage, and fibrosis. Once all the sutures are made, the strictured tubular structure or organ will naturally be pulled open from its original strictured position (see FIG. 42), thus providing for a wider drainage pathway at that particular site.

In an embodiment of the method, a balloon dilator catheter may be used to assist in the placement of the scaffold at the site of stricture. A balloon dilator catheter is advanced to the site of the stricture in the tubular structure or organ. The balloon dilator catheter is used to dilate the tubular structure or organ at the site of stricture by inflating the balloon dilator catheter. Once placement and suturing is complete, the balloon dilator is deflated and retracted from the tubular structure or organ.

Confirming restoration of flow to the tubular structure or organ comprises the steps of injecting a contrast dye into the tubular structure or organ and observing the flow of the contrast dye through the tubular structure or organ using a fluoroscopy x-ray apparatus.

The foregoing external scaffolds for expanding strictures in tubular organs and their use provide the advantages of being permanent, not involving placement of a device within the lumen of the tubular structure or organ, decreased risk of morbidity, decreased recovery time, and decreased hospitalization time. Furthermore, the resulting relief of strictures is immediately evident, which reduces the need for prolonged medical supervision.

The foregoing external scaffolds are particularly useful for treating multiple strictures on the same tubular structure or organ. For example, a single stricture in a ureter may be surgically removed in an ureteroureterostomy wherein the strictured section is completely cut and removed, and the remaining ends rejoined. However, this method cannot be used to treat multiple strictures because making complete transverse cuts on the same ureter causes the section of tissue between the two cuts to necrose, or die, due to lack of blood supply. In such a case, transplantation is required. The foregoing external scaffolds circumvent this problem and allow for the facile relief of multiple strictures.

In another example, while a single stricture that spans a short portion of an affected ureter can be removed by an ureteroureterostomy, a single stricture that spans a long portion of an affected ureter cannot be removed by an ureteroureterostomy because it may not be possible to stretch and rejoin the remaining ends. In such a case, transplantation is required. The foregoing external scaffolds circumvent this problem.

The features of the present invention disclosed in the specification, the claims and/or the drawings may both separately and in any combination thereof be material for realizing the invention in various forms thereof.

Even though numerous characteristics and advantages of the present invention have been set forth in the foregoing description, together with details of the structure and feature of the invention, the present disclosure is illustrative only. The details described hereinabove should not be viewed as limitations to the claims that follow.

Changes may be made in details by those having ordinary skill in the art without departing from the spirit and scope of the present invention.

1. An external scaffold for expanding a stricture in the lumen of a tubular structure or organ in a mammal comprising:
one or more open frames,
each open frame comprising a plurality of frame indentations, an inward-facing surface, an outward-facing surface, and an opening between opposite first and second ends;
said open frame has an inner frame width between about 0.25 inches and about 2.5 inches, and a cross-sectional width between about 0.065 inches and about 0.25 inches;
said frame indentations each have a frame indentation width between about 0.065 inches and about 0.125 inches, and a frame indentation depth not exceeding one-half of the cross-sectional width of said open frame;
said opening between first and second ends has an opening width between about 0 inches and about 0.25 inches; and
when said external scaffold comprises two or more open frames,
each open frame is juxtaposed to an adjacent open frame along the scaffold axis; and
each open frame is separated from an adjacent open frame by one or more cylindrical spacers, the cylindrical spacers comprising a plurality of spacer indentations; and
each open frame forms a twist angle with an adjacent open polygonal or toroidal frame between about 0° and about 90°.

2. The external scaffold according to claim 1, wherein the one or more open frames are open polygonal or toroidal frames, or a combination thereof.

3. The external scaffold according to claim 2, comprising one or more open polygonal frames, each polygonal frame independently selected from the group consisting of a regular polygon and a Reuleaux polygon.

4. The external scaffold according to claim 2, comprising one or more open toroidal frames.

5. (canceled)

6. The external scaffold according to claim 2, wherein the inner frame width is between about 0.25 inches and about 0.75 inches.

7. The external scaffold according to claim 2, wherein the frame indentations are each arc-shaped, V-shaped, or a combination thereof.

8. (canceled)

9. The external scaffold according to claim 2, wherein the opening width is between about 0.0625 inches and about 0.25 inches.

10. (canceled)

11. The external scaffold according to claim 2, wherein the inward-facing surface further comprises 3 or more spikes protruding from said inward-facing surface, wherein each spike comprises
a first segment connected directly to the inward-facing surface, the first segment perpendicular to the scaffold axis, and
a second segment connected directly to the end of the first segment that is not connected to the inward-facing surface, wherein the angle formed between said first and second segment is about 90°; and
wherein said second segment comprises a needlepoint at the end not connected to the first segment.

12.-14. (canceled)

15. The external scaffold according to claim 2, wherein the opposite first and second ends of the one or more open polygonal or toroidal frames further comprise interchangeable and complementary clasping means capable of being engaged in a locked position.

16. The external scaffold according to claim 1, comprising two or more open polygonal or toroidal frames.

17. The external scaffold according to claim 16, wherein said spacer indentations are each arc-shaped, V-shaped, or a combination thereof.

18. The external scaffold according to claim 16, comprising 2 or more open toroidal frames, wherein each open toroidal frame is an open torus frame.

19.-24. (canceled)

25. An adjustable external scaffold for expanding a stricture in the lumen of a tubular structure or organ in a mammal comprising:
one or more arcuate assemblies,
each arcuate assembly comprising
a first semicircular arc and a second semicircular arc pivotally connected at a hinge,
said first semicircular arc and a second semicircular arc each comprising a plurality of indentations on the outward-facing surface;
said hinge comprising a clicking means whereby the first semicircular arc and the second semicircular are held in place in the absence of force and capable of opening or closing in the presence of force.

26.-27. (canceled)

28. A method for expanding a stricture in the lumen of a tubular structure or organ in a mammal, the method comprising the steps of:
a) identifying the location of a stricture in the lumen of a tubular structure or organ in a mammal,
b) placing the scaffold according to claim 1 at the site of said stricture,
c) suturing in place said scaffold at the site of said stricture using sutures, and
d) confirming restoration of flow to said tubular structure or organ;
thereby expanding a stricture in the lumen of a tubular structure or organ in a mammal.

29.-30. (canceled)

31. The method according to claim 28, wherein the step of identifying the location of a stricture in the lumen of a tubular structure or organ in a mammal comprises the steps of:
a1) injecting a contrast dye near the lumen of a tubular structure or organ in a mammal,
a2) observing the flow of the contrast dye through said tubular structure or organ using a fluoroscopy x-ray apparatus, and
a3) noting the location of a stricture in said tubular structure or organ.

32. The method according to claim 28, wherein the step of placing the scaffold according to claim 1 at the site of said stricture comprises the steps of:
b1) providing said scaffold and placing said scaffold over the site of stricture in the tubular structure or organ, and
b2) connecting the muscle layer of said tubular structure or organ to said scaffold at the site of said stricture by making one initial suture.

33. The method according to claim 28, wherein the step of suturing in place said scaffold at the site of said stricture using sutures comprises applying sutures in a substantially symmetrical pattern.

34. The method according to claim 28, wherein the step of confirming restoration of flow to said tubular structure or organ comprises the steps of:
d1) injecting a contrast dye into said tubular structure or organ,
d2) observing the flow of the contrast dye through said tubular structure or organ using a fluoroscopy x-ray apparatus.

35. The method according to claim 28, further comprising the steps of:
e1) providing a balloon dilator catheter and advancing said balloon dilator catheter to the site of stricture in said tubular structure or organ;
e2) dilating said tubular structure or organ at the site of stricture by inflating said balloon dilator catheter; and
e3) deflating and retracting said balloon dilator catheter from said tubular structure or organ.

36. The method according to claim 28, wherein the tubular structure or organ is the esophagus, the pylorus, the small or large intestines, a bile duct, a vein, an artery, a fallopian tube, or a ureter.
